(19) United States
(12) Patent Application Publication
(10) Pub. No.: US 2025/0152500 A1
(43) Pub. Date: May 15, 2025

(54) METHOD FOR PREPARING FERMENTED VEGETABLE OIL BY MEANS OF SKIN FLORA, AND COSMETIC COMPOSITION COMPRISING SAME

(71) Applicants: COSMAX AB Inc., Seoul (KR); COSMAX, INC., Gyeonggi-do (KR)

(72) Inventors: Kwang Nyeon KIM, Seoul (KR); Hyun Tak HAN, Seoul (KR); Ju Hyun SON, Seoul (KR); Sa Rang CHO, Seoul (KR); Ju Yun KIM, Seoul (KR); Tae Hyun KIM, Seoul (KR); Sugyeong JEONG, Seoul (KR); Seok Kyun YUN, Gyeonggi-do (KR); Yae Rin LEE; Seung Hyun KANG, Seoul (KR); Myeong Sam PARK, Seoul (KR)

(21) Appl. No.: 18/707,873

(22) PCT Filed: Sep. 14, 2022
(86) PCT No.: PCT/KR2022/013663

§ 371 Date: May 6, 2024

(30) Foreign Application Priority Data
Nov. 9, 2021 (KR) 10-2021-0152923

(51) Int. Cl. A61K 8/99; A61Q 19/00; C12N 1/20; C12P 7/6445; C12R 1/01

(52) U.S. Cl. CPC A61K 8/99; A61Q 19/00; C12N 1/205; C12P 7/6445; A61K 2800/85; C12R 2001/01

(57) ABSTRACT

The present invention relates to a cosmetic composition in which lipid fermentation metabolites are produced by fermenting vegetable oil with skin flora, and a method for producing the same. According to the present invention, lipid fermentation metabolites similar to skin lipids can be produced by fermenting vegetable oil with skin flora. Further, thereby, the skin affinity and skin barrier-protecting effect of the fermented vegetable oil can be maximized.

### TECHNICAL FIELD

The present invention relates to a cosmetic composition that has improved skin affinity and absorption by fermenting vegetable oil with skin flora, and thus exhibits a skin barrier function-protecting effect, and a method for producing the same.

### BACKGROUND ART

The skin is composed of the epidermis, dermis, and subcutaneous fat layers. Among them, the epidermis exists in the outermost layer, serves as a protective film for the skin, and is responsible for the immune function of the skin. The skin is inhabited by skin flora, and these microorganisms are mainly found in the outermost layer of the epidermis and the upper part (upper layer) of hair follicles. There are about 1,000 kinds of microorganisms as the skin flora, and among them, aerobic microorganisms can secrete lipolytic enzymes that can utilize lipids in the epidermal layer.

The epidermal layer of the skin is mainly composed of keratinocytes, lipid components are formed between the keratinocytes, and the outermost layer is composed of protein. The stratum corneum of the epidermis contains about 10 to 20% of moisture and exists in the outermost layer of the human body, thereby suppressing the evaporation of moisture to the outside of the body while blocking the excessive penetration of contaminants from the outside. These stratum corneum surfaces can be surrounded by a thin natural protective film made of sebum from sebaceous glands and sweat from sweat glands, thereby preventing the evaporation of moisture. The cells that make up this stratum corneum contain a high concentration of natural moisturizing factor (NMF), a water-soluble component, which helps maintain skin flexibility and appropriate moisture. For example, substances such as amino acids are not only water-soluble, but also effectively bind with moisture, thereby preventing the skin moisture from drying out.

The stratum corneum of the skin may be damaged due to various causes such as aging, and stress caused by various stimuli such as artificial temperature control of cooling/heating, changes in the external environment such as environmental pollution, or changes in lifestyle patterns. In addition, as moisture in the stratum corneum decreases, the skin becomes dry, the skin surface becomes rough, and the skin loses its luster, which has a negative effect on the skin in terms of beauty and skin health.

Meanwhile, vegetable oils are known to contain a large amount of active substances that have a beneficial effect on the skin, but are difficult to be absorbed into the skin and have characteristics such as oil-specific heavy feeling of use and unique odor, so their use in cosmetics is limited.

The present invention seeks to provide a composition that can improve skin health by utilizing flora beneficial to the skin and vegetable oil, and a method for producing the same.

### DISCLOSURE OF THE INVENTION

### Technical Goals

An object of the present invention is to provide a cosmetic composition that has improved skin affinity and adhesion due to oil fermentation metabolites of skin flora, produced by fermenting vegetable oil with skin flora, and thus exhibits a skin barrier-protecting effect, and a method for producing the same.

### Technical Solutions

In order to solve the above problem, the present invention provides a method for producing a cosmetic composition containing vegetable oil, the method including:
a seed culture step of producing a seed culture by culturing microorganisms with deposit number of KCCM 11843P (Korean Culture Center of Microorganisms (Overseas), 2016.06.08) as skin flora;
a preculture step of culturing the seed culture to produce a preculture; and
a main fermentation step of producing a fermented product by adding the preculture to a medium containing vegetable oil.

According to one embodiment, the medium of the seed culture step or the medium of the preculture step may contain casein hydrolyzate, yeast extract, glucose, soluble starch, K₂HPO₄, sodium pyruvate, casein peptone, and MgSO₄.

Specifically, for example, the medium of the seed culture step or the medium of the preculture step may contain 0.1 to 5 g/L of casein hydrolysate, 0.1 to 10 g/L of yeast extract, 0.1 to 10 g/L of glucose, 0.1 to 5 g/L of soluble starch, 0.1 to 5 g/L of K₂HPO₄, 0.05 to 5 g/L of sodium pyruvate, 0.1 to 5 g/L of casein peptone, and 0.01 to 1 g/L of MgSO₄.

According to one embodiment, the medium of the main fermentation step may contain glycerol, yeast extract, casein peptone, K₂HPO₄, KH₂PO₄, MgSO₄·7H₂O, NaCl, NH₂SO₄, KNO and FeSO₄.

Specifically, for example, the medium of the main fermentation step may contain 0.1 to 50 g/L of glycerol, 0.1 to 50 g/L of yeast extract, 0.1 to 50 g/L of casein peptone, 0.01 to 5 g/L of K₂HPO₄, 0.01 to 3 g/L of KH₂PO₄, 0.005 to 0.5 g/L of MgSO₄·7H₂O, 0.1 to 10 g/L of NaCl, 0.01 to 5 g/L of NH₂SO₄, 0.01 to 1 g/L of KNO, 0.001 to 0.1 g/L of FeSO₄, and 50 to 900 g/L of vegetable oil.

According to one embodiment, the seed culture step may include shaking culturing under aerobic conditions of 15 to 35° C.,
the preculture step may include adding 10 to 500 g/L of the seed culture and culturing under aerobic conditions of 15 to 35° C., 5 to 20 NL/min, and 10 to 100 rpm, and
The culture in the preculture step may be terminated when the spectrophotometric absorbance of 5% diluted culture solution at a wavelength of 600 nm is 0.2 to 0.8.

According to one embodiment, the main fermentation step may include adding 1 to 30% by weight of the preculture to the main fermentation medium and fermenting it for 30 to 150 hours under aerobic conditions of 15 to 35° C., 100 to 500 NL/min, and 400 to 800 rpm.

According to another embodiment of the present invention, there is provided a cosmetic composition containing vegetable oil prepared by the method described above.

Specific matters of other embodiments according to the present invention are presented in the detailed description below.

### Advantageous Effects

The cosmetic composition according to the present invention can have improved skin affinity, adhesion, and skin absorption rate by fermenting vegetable oil with skin flora, thereby maximizing beneficial effects on the skin, such as skin barrier protection.

### BRIEF DESCRIPTION OF DRAWINGS

FIG. 1 is a graph showing changes in pH and viable cell count according to main fermentation time.

FIG. 2 is a photograph of a composition according to Example 3 observed with the naked eye.

FIG. 3 is a chromatogram showing the results of TLC analysis.

FIG. 4 is a photograph observed with the naked eye confirming emulsification activity.

FIG. 5 is a chromatogram showing the results of TLC analysis of polar lipids.

### BEST MODE FOR CARRYING OUT THE INVENTION

Although the present invention may be modified in various ways and have various embodiments, specific embodiments will be exemplified and described in detail. However, the present invention is not intended to be limited to the specific embodiments, and should be understood to include all modifications, equivalents, and substitutes included in the spirit and technical scope of the present invention. In describing the present invention, if it is determined that detailed descriptions of related known technologies may obscure the gist of the present invention, the detailed description thereof will be omitted.

Hereinafter, a cosmetic composition containing vegetable oil using skin flora according to the present invention, and a method for producing the same will be described in detail.

In the present invention, vegetable oil was fermented with skin flora which has a beneficial effect on the skin and coexists among the various microorganisms that inhabit the skin. Lipid fermentation metabolites produced by fermentation provide a composition with improved effective effects on the skin.

Specifically, the present invention provides a method for producing a cosmetic composition containing vegetable oil, the method including:
a seed culture step of producing a seed culture by culturing microorganisms with deposit number of KCCM 11843P (Korean Culture Center of Microorganisms (Overseas), 2016.06.08) as skin flora;
a preculture step of culturing the seed culture to produce a preculture; and
a main fermentation step of producing a fermented product by adding the preculture to a medium containing vegetable oil.

The skin flora of the present invention is a strain of Epidermidibacterium keratini sp.

The vegetable oil used in the present invention is not particularly limited as long as it is not cytotoxic. Specifically, for example, the vegetable oil may include one or more of edible or human-friendly oils such as macadamia oil, sunflower seed, grape seed, canola, rice germ, olive, soybean, argan, brown rice, perilla seed, sesame seed, almond, peanut, corn, red ginseng, avocado, macadamia, coconut, rose hip, vitamin tree seed, shea tree fruit, oil palm, bergamot fruit, camellia seed, safflower seed, apricot seed, poppy seed, evening primrose seed, castor seed, green tea seed, meadowfoam seed, flax seed and hemp seed, but is not particularly limited thereto.

According to one embodiment, the medium of the seed culture step or the medium of the preculture step may contain casein acid hydrolyzate, yeast extract, glucose, soluble starch, K₂HPO₄, sodium pyruvate, casein peptone, and MgSO₄.

Specifically, for example, the medium of the seed culture step or the medium of the preculture step may contain 0.1 to 5 g/L, such as 0.1 to 3 g/L, or 0.3 to 1 g/L, of casein acid hydrolyzate, 0.1 to 10 g/L, such as 0.1 to 5 g/L, or 0.1 to 3 g/L, or 0.3 to 1 g/L, of yeast extract, 0.1 to 10 g/L, such as 0.1 to 5 g/L, or 0.1 to 3 g/L, or 0.1 to 1 g/L, of glucose, 0.1 to 5 g/L, such as 0.1 to 3 g/L, or 0.1 to 1 g/L, of soluble starch, 0.1 to 5 g/L, such as 0.1 to 3 g/L, or 0.1 to 1 g/L, of K₂HPO₄, 0.05 to 5 g/L, such as 0.05 to 3 g/L, or 0.05 to 1 g/L, or 0.1 to 0.5 g/L, of sodium pyruvate, 0.1 to 5 g/L, such as 0.1 to 3 g/L, or 0.1 to 1 g/L, of casein peptone, and 0.01 to 1 g/L, such as 0.01 to 0.5 g/L, or 0.01 to 0.1 g/L, of MgSO₄.

According to one embodiment, the medium of the main fermentation step may contain glycerol, yeast extract, casein peptone, K₂HPO₄, KH₂PO₄, MgSO₄·7H₂O, NaCl, NH₂SO₄, KNO and FeSO₄.

Specifically, for example, the medium of the main fermentation step may contain 0.1 to 50 g/L, such as 1 to 30 g/L, or 1 to 20 g/L, or 1 to 10 g/L, of glycerol, 0.1 to 50 g/L, such as 1 to 30 g/L, or 1 to 20 g/L, or 1 to 10 g/L, of yeast extract, 0.1 to 50 g/L, such as 0.1 to 30 g/L, or 0.1 to 5 g/L, of casein peptone, 0.01 to 5 g/L, such as 0.01 to 3 g/L, or 0.01 to 1 g/L, or 0.1 to 1 g/L, of K₂HPO₄, 0.01 to 3 g/L, such as 0.01 to 1 g/L, or 0.1 to 1 g/L, of KH₂PO₄, 0.005 to 0.5 g/L, such as 0.005 to 0.1 g/L, or 0.01 to 0.05 g/L, of MgSO₄·7H₂O, 0.1 to 10 g/L, such as 0.1 to 5 g/L, or 0.5 to 3 g/L, of NaCl, 0.01 to 5 g/L, 0.01 to 3 g/L, or 0.1 to 1 g/L, of NH₂SO₄, 0.01 to 1 g/L, such as 0.05 to 0.5 g/L, or 0.05 to 0.3 g/L, of KNO, 0.001 to 0.1 g/L, such as 0.001 to 0.05 g/L, or 0.005 to 0.03 g/L, of FeSO₄, and 50 to 900 g/L, such as 100 to 800 g/L, or 300 to 600 g/L, of vegetable oil.

According to one embodiment, the seed culture step may include shaking culturing under aerobic conditions of 15 to 35° C., such as 20 to 30° C.

According to one embodiment, the preculture step may include adding 10 to 500 g/L, such as 50 to 300 g/L, 50 to 200 g/L, 50 to 150 g/L, of the seed culture to the preculture medium and culturing under aerobic conditions of 15 to 35° C., such as 15 to 25° C., 5 to 20 NL/min, such as 10 to 20 NL/min, and 10 to 100 rpm, such as 30 to 80 rpm.

In addition, the culture in the preculture step may be terminated when the spectrophotometric absorbance of the 5% culture solution at a wavelength of 600 nm is 0.2 to 0.8, such as 0.2 to 0.4.

According to one embodiment, the main fermentation step may include adding 1 to 30 wt %, such as 2 to 20 wt %, or 5 to 15 wt %, of the preculture to the main fermentation medium and fermenting it for 30 to 150 hours, such as 50 to 100 hours, or 60 to 80 hours under aerobic conditions of 15 to 35° C., such as 15 to 25° C., 100 to 500 NL/min, such as 200 to 400 NL/min, and 400 to 800 rpm, such as 500 to 700 rpm.

According to one embodiment, separation and purification steps may be further included after the main fermentation step. For example, a step of centrifuging the fermented product to separate the oil layer and the water layer, and then recovering the oil layer may be included. In addition, filtration using a filter can be applied by adding and stirring MgSO₄ to remove residual moisture and impurities, but the method applied for purification is not limited to the above.

According to one embodiment, the cosmetic composition of the present invention may include components commonly used in cosmetic compositions, such as stabilizers, solubilizers, vitamins, pigments, flavors, adjuvants, and carriers.

In addition, it may be prepared in any formulation that is commonly manufactured in the art and is dermatologically applicable.

‘Dermatologically applicable’ may mean that a composition can have an effective action that is relatively non-toxic and harmless to the subject to which it is applied, and may include an external agent applicable to the skin, wherein side effects resulting from the composition do not reduce the efficacy of the active ingredients, do not cause serious irritation to the applied subject, and do not impair the activity and properties of the active ingredients. The dermatologically applicable cosmetic composition of the present invention may be formulated, for example, as a solution, suspension, emulsion, emulsified liquid, paste, gel, pack, cream, lotion, powder, soap, surfactant-containing cleansing, oil, powder foundation, emulsion foundation, wax foundation, spray, and hair cosmetic, but is not limited thereto.

Specifically, it may be manufactured as a formulation of skin lotion, skin softener, skin toner, astringent, lotion, gel, milk lotion, moisture lotion, nutritional lotion, massage cream, nutritional cream, moisture cream, hand cream, foundation, essence, ampoule, nutritional essence, pack, soap, hair shampoo, foot shampoo, cleansing foam, cleansing lotion, cleansing cream, body lotion, and body cleanser.

Hereinafter, examples of the present invention will be described in detail so that those skilled in the art can easily implement the present invention. However, the present invention may be embodied in a variety of forms and is not limited to the examples described herein.

### Example: Preparation of a Cosmetic Composition Containing Fermented Vegetable Oil Using Skin Flora

### Seed Culture

As skin flora, a microorganism with deposit number of KCCM 11843P (Korean Culture Center of Microorganisms (Overseas), 2016.06.08) was seed cultured. It was inoculated into R2A (BD Difco, USA) medium, and shaking culture was performed under aerobic conditions at 25° C. to prepare a seed culture. Specifically, the medium has a composition of 0.5 g/L of casein acid hydrolysate, 0.5 g/L of yeast extract, 0.5 g/L of glucose, 0.5 g/L of soluble starch, 0.3 g/L of K₂HPO₄, 0.3 g/L of sodium pyruvate, 0.5 g/L of casein peptone, and 0.05 g/L of MgSO₄.

### Preculture

100 g/L of the seed culture was added to a medium having the same composition as the seed culture medium, and cultured under aerobic conditions at 20° C., 15 NL/min, and 50 rpm to prepare a preculture. In addition, for the preculture, the time point of the main fermentation was set by turbidity measurement. Specifically, the preculture was sampled and diluted to 5% with 0.85% NaCl solution, and then the absorbance was measured at a wavelength of 600 nm using a spectrophotometer (BioTek, USA). The end point of preculture and the inoculation point of main fermentation were set based on the absorbance range of 0.2 to 0.4.

### Main Fermentation

The main fermentation medium was as shown in Table 1. When the temperature of each medium according to Table 1 is 20° C., 10% by weight of the preculture is inoculated, and fermented under aerobic conditions at 20° C., 300 NL/min, and 600 rpm to produce a fermented product (fermented oil).

TABLE 1

| Classification | Ex- | Ex- | Ex- | Ex- | Ex- | Ex- |
|---|---|---|---|---|---|---|
| (g/L) | ample 1 | ample 2 | ample 3 | ample 4 | ample 5 | ample 6 |
| Glycerol | 7.5 | 7.5 | 7.5 | 7.5 | 7.5 | 7.5 |
| Yeast extract | 5.0 | 5.0 | 5.0 | 5.0 | 5.0 | 5.0 |
| Casein | 1.0 | 1.0 | 1.0 | 1.0 | 1.0 | 1.0 |
| peptone | | | | | | |
| K₂HPO₄ | 0.45 | 0.45 | 0.45 | 0.45 | 0.45 | 0.45 |
| KH₂PO₄ | 0.3 | 0.3 | 0.3 | 0.3 | 0.3 | 0.3 |
| MgSO₄•7H₂O | 0.02 | 0.02 | 0.02 | 0.02 | 0.02 | 0.02 |
| NaCl | 1.0 | 1.0 | 1.0 | 1.0 | 1.0 | 1.0 |
| NH₂SO₄ | 0.5 | 0.5 | 0.5 | 0.5 | 0.5 | 0.5 |
| KNO | 0.1 | 0.1 | 0.1 | 0.1 | 0.1 | 0.1 |
| FeSO₄ | 0.01 | 0.01 | 0.01 | 0.01 | 0.01 | 0.01 |
| Macadamia | 500.0 | — | — | — | — | — |
| oil | | | | | | |
| Sunflower | — | 500.0 | — | — | — | — |
| seed oil | | | | | | |
| Brown | — | — | 500.0 | — | — | — |
| rice oil | | | | | | |
| Grape | — | — | — | 500.0 | — | — |
| seed oil | | | | | | |
| Apricot | — | — | — | — | 500.0 | — |
| seed oil | | | | | | |
| Olive | — | — | — | — | — | 500.0 |
| oil | | | | | | |
| Purified | 484.12 | 484.12 | 484.12 | 484.12 | 484.12 | 484.12 |
| water | | | | | | |

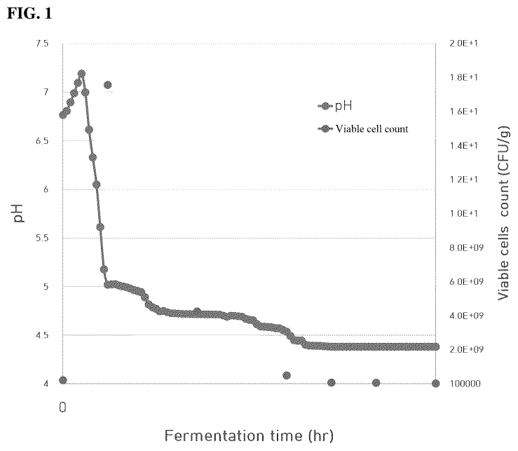

In addition, the changes in pH and viable cell count according to the main fermentation time in Example 1 are graphically shown in FIG. 1. From the graph in FIG. 1, it was confirmed that it was most efficient to proceed with the main fermentation for 72 hours.

### Separation and Purification

In order to obtain fermented oil of the fermented product, the fermented product was centrifuged to separate an oil layer. The oil layer was recovered, and 100 g/L of MgSO₄ was added and then stirred to remove remaining moisture and impurities. After stirring for 2 hours, it was filtered using a filter pad (CH-ST-150, Hyundai Micro, Korea) in a filter press. The filtered oil was finally sterilized and filtered (0.2 μm filter) to obtain fermented oil.

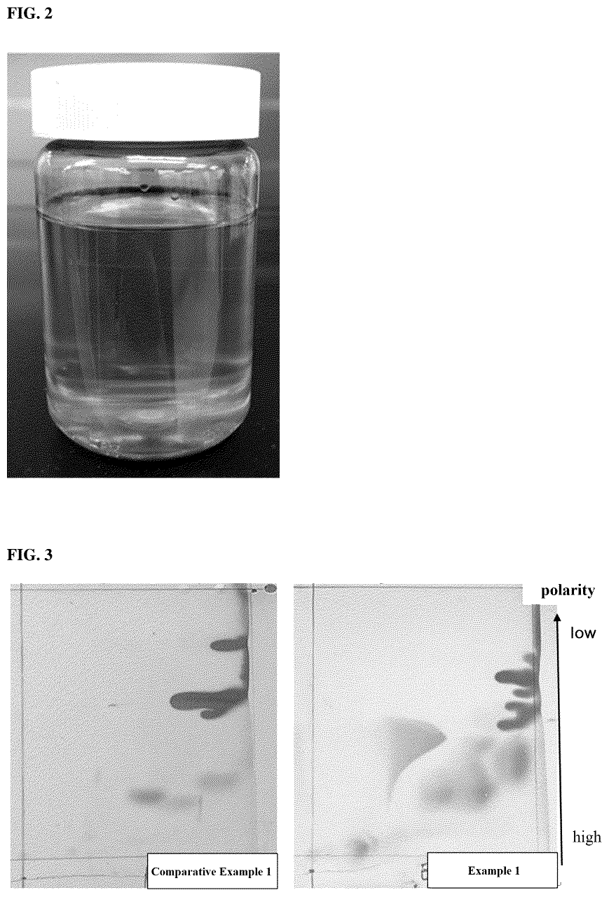

A photograph of the composition according to Example 3 observed with the naked eye was shown in FIG. 2.

### Comparative Example 1

Unfermented brown rice oil was used.

### Experimental Example 1: TLC Analysis

1 ul of the sample diluted 20% in chloroform was added dropwise to a silica gel TLC plate (TLC Silica gel 60, Merck; 5 cm×6.5 cm), and developed to the end point with a mobile phase solvent containing hexane, di-ethyl ether, and acetone (7:3:0.1, v/v). It was secondarily developed to the end point with a mobile phase solvent containing chloroform, methanol, and acetone (12:0.5:0.2, v/v) on the naturally dried plate. 10% sulfuric acid mixed with ethanol was sprayed on the naturally dried plate, dried, and heated at 100° C. for 5 minutes to visualize the spot. The results of comparing Comparative Example 1 and Example 1 are shown in FIG. 3. As shown in FIG. 3, spots (components) with higher lipid polarity were colored in Example 1 compared to Comparative Example 1. In the case of Example 1, the skin absorption rate of the composition can be increased by improving the polarity of the oil, and the skin barrier-strengthening function can be improved due to the absorption of the lipid component into the skin.

### Experimental Example 2: Confirmation of Emulsification Activity

In order to confirm the emulsification activity of the composition, before and after the fermentation of Example 3 were compared. Specifically, purified water was mixed at a ratio of 1:2 in Comparative Example 1 and Example 3, and the temperature was raised to 60° C.

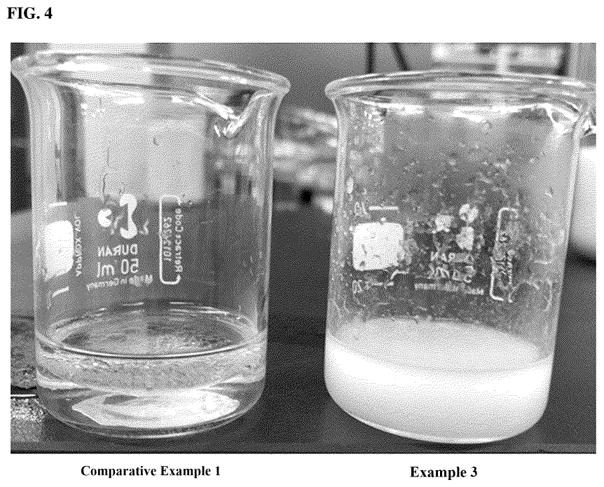

Thereafter, the results of cooling and observing the emulsification state are shown in FIG. 4. In Comparative Example 1 of FIG. 4, the oil layer and the aqueous layer were separated, and the layers were clearly distinguished. On the other hand, it was confirmed in Example 3 that the oil layer and the aqueous layer were evenly emulsified.

### Experimental Example 3: Confirmation of the Production of Amino Groups in Lipid Fermentation Metabolites

For the lipid fermentation metabolites of Example 3, it was confirmed using a Ninhydrin reaction test method whether an amino group was produced for each fermentation time. Specifically, the culture solution of Example 3 was treated with 1N HCl and then centrifuged to recover the lipid layer. The recovered lipid layer was neutralized with sodium bicarbonate (sodium hydrogen carbonate) and extracted with chloroform-methanol (2:1, v/v) to recover the solvent layer and obtain a sample to be used for analysis. The obtained sample was reacted with 0.1% Nihydrin solution (solvent: ethanol). The mixing ratio was sample 2:0.1% Ninhydrin solution 1, and the reaction was performed under the condition of heating in a 90° C. water bath for 10 minutes. Thereafter, it was left to cool at room temperature for 10 minutes, and the absorbance was measured at 570 nm using a spectrophotometer (BioTek, USA).

TABLE 2

| Example | 0 | 12 | 36 | 60 | 72 | 84 | 100 |
|---|---|---|---|---|---|---|---|
| 3 | hours | hours | hours | hours | hours | hours | hours |
| O.D | 0.00 | 0.10 | 0.22 | 0.46 | 0.64 | 0.63 | 0.63 |
| 570 nm | | | | | | | |

As a result of Ninhydrin reaction of Example 3 for each fermentation time, the highest reactivity was shown at 72 hours.

### Experimental Example 4: TLC Analysis of Polar Lipids

The fermentation metabolites of Example 3 were subjected to TLC analysis to confirm polar lipids. Specifically, the culture solution of Example 3 was treated with 1N HCl and then centrifuged to recover the lipid layer. The recovered lipid layer was neutralized with sodium bicarbonate (sodium hydrogen carbonate) and extracted with chloroform-methanol (2:1, v/v) to recover the solvent layer and obtain a sample to be used for analysis.

1 ul of the sample diluted 20% in chloroform was added dropwise to a silica gel TLC plate (TLC Silica gel 60, Merck; 5 cm×6.5 cm), and developed to the end point with a mobile phase solvent containing chloroform, methanol, and water (7.5:2.5:0.5, v/v). Visualization was achieved by developing color for 5 minutes at room temperature under light-blocking conditions using molybdenum blue spray reagent (Sigma aldrich) as a coloring agent.

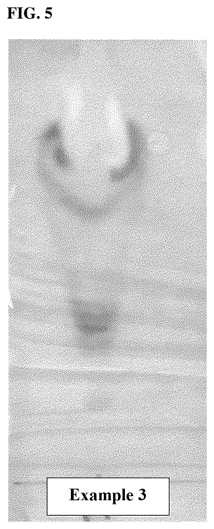

As shown in FIG. 5, blue spots were confirmed as a result of staining with Molybdenum blue, which develops polar lipids. As a result, it could be confirmed that highly polar lipids or phospholipids are produced in the fermentation metabolites of vegetable oil with skin bacteria.

As described above, the composition containing fermented vegetable oil according to the present invention can have improved skin absorption by producing a highly polar lipid component. In addition, it has improved emulsion stability and thus has satisfactory skin applicability and feeling of use, making it suitable as a cosmetic composition.

The specific contents of the present invention have been described in detail above, and for those skilled in the art, these specific descriptions are merely preferred embodiments, and thus the scope of the present invention is not limited to the specific embodiments described above.

[Microorganism Deposit Certificate]

1. A method for producing a cosmetic composition containing vegetable oil, the method including:
a seed culture step of producing a seed culture by culturing microorganisms with deposit number of KCCM 11843P (Korean Culture Center of Microorganisms (Overseas), 2016.06.08) as skin flora;
a preculture step of culturing the seed culture to produce a preculture; and
a main fermentation step of producing a fermented product by adding the preculture to a medium containing vegetable oil.

2. The method for producing a cosmetic composition containing vegetable oil according to claim 1, wherein the medium of the seed culture step or the medium of the preculture step contains casein hydrolyzate, yeast extract, glucose, soluble starch, K₂HPO₄, sodium pyruvate, casein peptone, and MgSO₄.

3. The method for producing a cosmetic composition containing vegetable oil according to claim 1, wherein the medium of the seed culture step or the medium of the preculture step contains 0.1 to 5 g/L of casein hydrolysate, 0.1 to 10 g/L of yeast extract, 0.1 to 10 g/L of glucose, 0.1 to 5 g/L of soluble starch, 0.1 to 5 g/L of K₂HPO₄, 0.05 to 5 g/L of sodium pyruvate, 0.1 to 5 g/L of casein peptone, and 0.01 to 1 g/L of MgSO₄.

4. The method for producing a cosmetic composition containing vegetable oil according to claim 1, wherein the medium of the main fermentation step contains glycerol, yeast extract, casein peptone, K₂HPO₄, KH₂PO₄, MgSO₄·7H₂O, NaCl, NH₂SO₄, KNO and FeSO₄.

5. The method for producing a cosmetic composition containing vegetable oil according to claim 1, wherein the medium of the main fermentation step contains 0.1 to 50 g/L of glycerol, 0.1 to 50 g/L of yeast extract, 0.1 to 50 g/L of casein peptone, 0.01 to 5 g/L of K₂HPO₄, 0.01 to 3 g/L of KH₂PO₄, 0.005 to 0.5 g/L of MgSO₄·7H₂O, 0.1 to 10 g/L of NaCl, 0.01 to 5 g/L of NH₂SO₄, 0.01 to 1 g/L of KNO, 0.001 to 0.1 g/L of FeSO₄, and 50 to 900 g/L of vegetable oil.

6. The method for producing a cosmetic composition containing vegetable oil according to claim 1, wherein the seed culturing step includes shaking culturing under aerobic conditions of 15 to 35° C.,
the preculture step includes adding 10 to 500 g/L of the seed culture and culturing under aerobic conditions of 15 to 35° C., 5 to 20 NL/min, and 10 to 100 rpm, and
the culture in the preculture step is terminated when the spectrophotometric absorbance of the 5% culture solution at a wavelength of 600 nm is 0.2 to 0.8.

7. The method for producing a cosmetic composition containing vegetable oil according to claim 1, wherein the main fermentation step includes adding 1 to 30% by weight of the preculture to the main fermentation medium and fermenting it for 30 to 150 hours under aerobic conditions of 15 to 35° C., 100 to 500 NL/min, and 400 to 800 rpm.

8. A cosmetic composition containing vegetable oil, produced by the method according to claim 1.
